(19) United States
(12) Patent Application Publication
(10) Pub. No.: US 2010/0057172 A1
(43) Pub. Date: Mar. 4, 2010

(54) Surgical apparel for covering an area of a patient's head and reducing heat loss

(76) Inventor: Sarah Hipps, Seattle, WA (US)

Correspondence Address: CHARLES J RUPNICK, PO BOX 46752, SEATTLE, WA, 98146, US

(21) Appl. No.: 12/590,297

(22) Filed: Nov. 5, 2009

Related U.S. Application Data
(63) Continuation of application No. 11/680,294, filed on Feb. 28, 2007.

(51) Int. Cl. A61F 7/00

(52) U.S. Cl. 607/110

(57) ABSTRACT

Surgical apparel comprising a substantially conically shaped shell having a narrower closed end and a wider open end dimensioned to fit over a crown area of a head of a surgical patient is described herein. The shell may include one or more layers of fabric having a thermal transfer property adapted to complement the closed end to reduce an amount of heat transfer from the head of the surgical patient to the ambient during a surgical procedure. Also, the fabric may be relatively inexpensive to enable disposal of the surgical apparel after a single use on the surgical patient.

### TECHNICAL FIELD

Embodiments relate to the fields of medical devices and apparel, in particular, to surgical apparel for covering an area of a surgical patient's head during a surgical procedure to reduce heat loss from the patient.

### BACKGROUND

Advances in surgical procedures and equipment have enabled greater success in a wide variety of surgeries. As a result, surgical patients enjoy far greater chances of having successful results to their surgeries than ever before. A variety of factors, however, continue to influence the effectiveness of a given procedure or piece of equipment in a given surgery. Among these factors is heat retention. Surgical patients losing body heat through diffusion and air flow tend to fare worse than surgical patients retaining body heat. One area of surgical patients that is susceptible to heat loss is the surgical patients' heads.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

Embodiments of the present invention will be described by way of exemplary embodiments, but not limitations, illustrated in the accompanying drawings in which like references denote similar elements, and in which:

FIG. 1 illustrates exemplary surgical apparel, in accordance with various embodiments;

FIG. 2 illustrates the surgical apparel being worn by a surgical patient, in accordance with various embodiments;

FIGS. 3a-3b illustrate surgical apparel having a flexible band or drawstring means surrounding a wider open end of the surgical apparel's shell, in accordance with various embodiments;

FIGS. 4a-4c illustrate top views of various embodiments of the surgical apparel;

FIGS. 5a-5b illustrate the narrower closed end of embodiments of the surgical apparel in a more open state to facilitate access to an area of the patient's head;

FIGS. 6a-6b illustrate surgical apparel having ear-covering shell attachments, in accordance with various embodiments;

FIGS. 7a-7b illustrate surgical apparel having an attached facial covering, in accordance with various embodiments;

FIGS. 8a-8b illustrate surgical apparel having both ear-covering shell attachments and an attached facial covering, in accordance with various embodiments;

FIG. 9 illustrates a cross section view of a shell of the surgical apparel, in accordance with various embodiments;

FIG. 10 illustrates surgical apparel dimensioned to cover all of a surgical patient's hair, in accordance with various embodiments;

FIG. 11 illustrates surgical apparel having an attached beard covering, in accordance with various embodiments;

FIG. 12 illustrates surgical apparel having an attached mustache covering, in accordance with various embodiments;

FIG. 13 illustrates surgical apparel having attached beard and mustache coverings, in accordance with various embodiments; and

FIG. 14 illustrates surgical apparel having an attached patient identifier, in accordance with various embodiments.

### DETAILED DESCRIPTION OF ILLUSTRATIVE EMBODIMENTS

Illustrative embodiments of the present invention include, but are not limited to, surgical apparel comprising a substantially conically shaped shell having a narrower closed end and a wider open end dimensioned to fit over a crown area of a head of a surgical patient. The shell may include one or more layers of fabric having a thermal transfer property adapted to complement the closed end to reduce an amount of heat transfer from the head of the surgical patient to the ambient during a surgical procedure. Also, the fabric may be relatively inexpensive to enable disposal of the surgical apparel after a single use on the surgical patient.

Various aspects of the illustrative embodiments will be described using terms commonly employed by those skilled in the art to convey the substance of their work to others skilled in the art. However, it will be apparent to those skilled in the art that alternate embodiments may be practiced with only some of the described aspects. For purposes of explanation, specific numbers, materials, and configurations are set forth in order to provide a thorough understanding of the illustrative embodiments. However, it will be apparent to one skilled in the art that alternate embodiments may be practiced without the specific details. In other instances, well-known features are omitted or simplified in order not to obscure the illustrative embodiments.

Further, various operations will be described as multiple discrete operations, in turn, in a manner that is most helpful in understanding the illustrative embodiments; however, the order of description should not be construed as to imply that these operations are necessarily order dependent. In particular, these operations need not be performed in the order of presentation.

The phrase “in one embodiment” is used repeatedly. The phrase generally does not refer to the same embodiment; however, it may. The terms “comprising,” “having,” and “including” are synonymous, unless the context dictates otherwise. The phrase “A/B” means “A or B”. The phrase “A and/or B” means “(A), (B), or (A and B)”. The phrase “at least one of A, B and C” means “(A), (B), (C), (A and B), (A and C), (B and C) or (A, B and C)”. The phrase “(A) B” means “(B) or (A B)”, that is, A is optional.

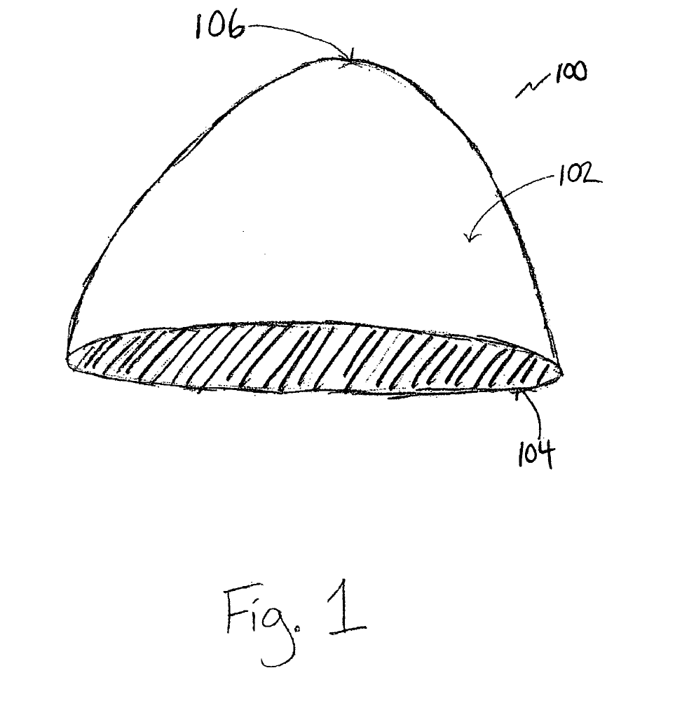

FIG. 1 illustrates exemplary surgical apparel, in accordance with various embodiments. As illustrated, surgical apparel 100 may comprise a substantially conically-shaped shell 102 having a wider open end 104 and a narrower closed end 106. In various embodiments, shell 102 may have one or more layers of fabric, the fabric having a thermal transfer property capable of reducing heat loss through a surgical patient's head to the ambient during a surgical procedure on a patient wearing the surgical apparel 100. The wider open end 104 of apparel 100 may have a sufficient size to allow apparel 100 to fit on the crown area of a surgical patient's head. The narrower closed end 106, in contrast, may be entirely closed, as shown, to prevent heat loss through air flow coming in contact with the patient's head. In some embodiments, the fabric and other materials comprising apparel 100 may be relatively inexpensive, allowing disposal of the surgical apparel 100 after a single use.

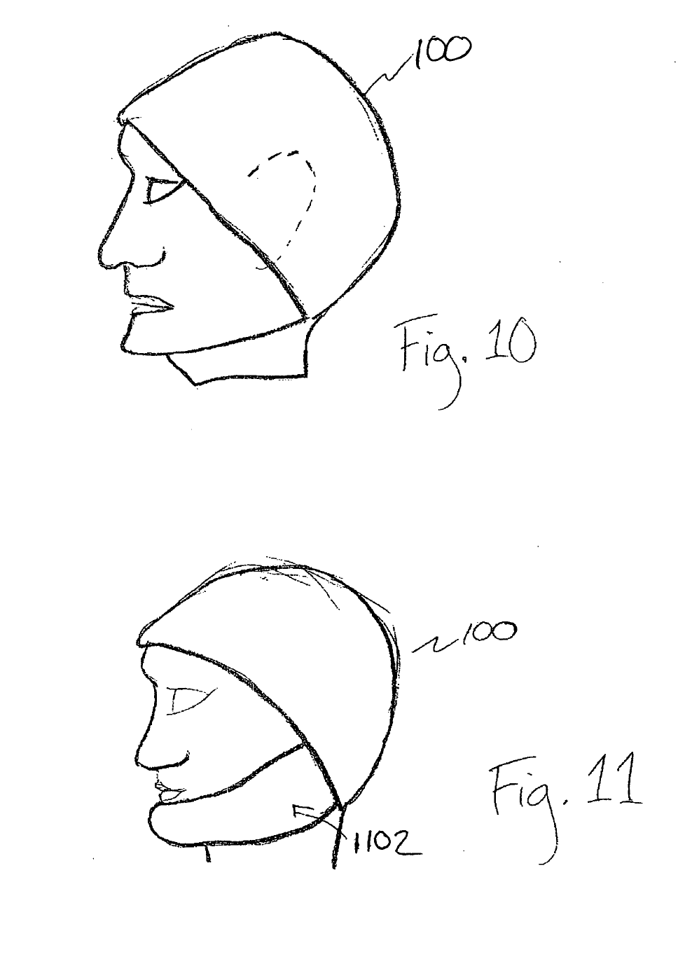

In various embodiments, the substantially conically-shaped shell 102 of apparel 100 may have a thermal transfer property such that the apparel 100 is capable of reducing heat loss through air flow contacting a patient's head and through diffusion into the ambient surrounding the surgical patient's head during a surgical procedure. The thermal transfer property may result from a fabric type of at least one layer of the one or more layers of fabric of shell 102, discussed in greater detail below, because, for example, of a low thermal transfer coefficient of a fabric type. In other embodiments, the thermal transfer property may result from a number of layers of fabric that, in combination, have a low thermal transfer coefficient, or from a thickness of one or more of shell 102's layers of fabric. Thus, the thermal transfer property may result from any conceivable type, number of layers, and thickness of fabrics known in the art such that the resultant shell 102 has a thermal transfer property capable of reducing heat loss from the patient's head to the ambient during a surgical procedure. Also, FIG. 9 illustrates in greater detail a cross-section view of shell 102 showing a plurality of layers of fabric of different types. Regardless of the number of layers and material types, however, shell 102 may be breathable, allowing some degree of airflow between the patient's head and the ambient.

In various embodiments, shell 102 may be comprised of any number of fabric layers and fabric types. Any one or more fabric types may be a fabric from one of four major fabric categories: animal textiles, plant textiles, mineral textiles, and synthetic textiles. Also, any layer of fabric may have a very light weight, as in the case of a gossamer fabric, or a very heavy weight, as in the case of a canvas fabric. In some embodiments, any one or more fabric types may be a fabric of a well known type, such as wool, silk, cotton, flax, jute, modal, polyester, acrylic, nylon, Lycra, spandex, Tactel, olefin fiber, Lurex, Ingeo, and Gore-Tex. A layer of fabric of shell 102 may also be a blend of one or more of these fabric types. Shell 102 may have layers of only one fabric type, or may have multiple layers, some of the layers having different fabric types from each other. As discussed above, each fabric may have a low thermal transfer coefficient, or a combination may be created resulting in a low thermal transfer coefficient. The number, thickness, and fabric types chosen for the layers of shell 102 may also vary with fabric costs to ensure that the shell 102 is relatively inexpensive and thus disposable after a single use.

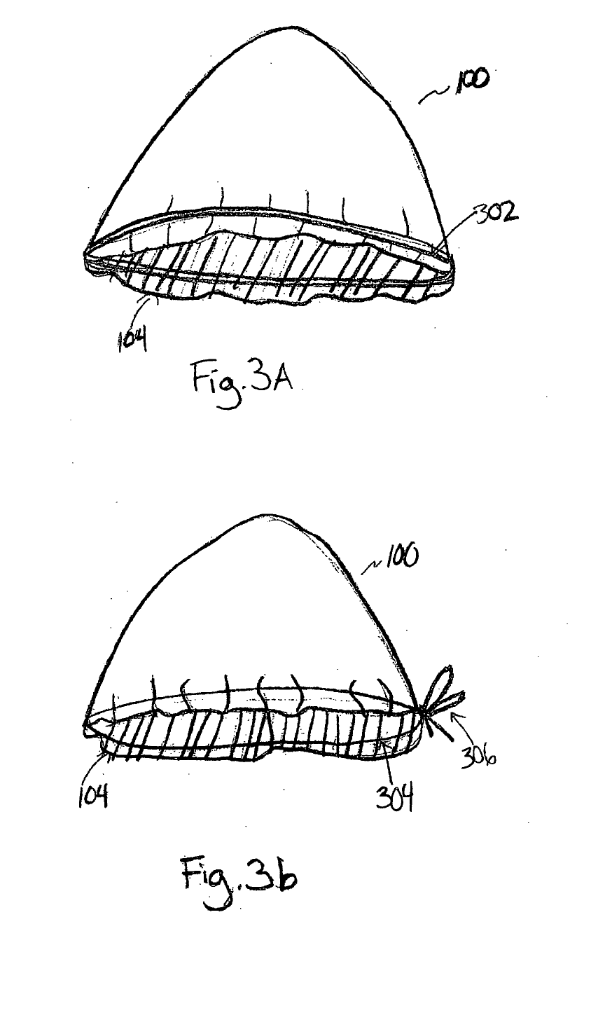

In some embodiments, wider open end 104 of apparel 100 may be sized to fit a specific patient head size. For example, apparel 100 may be sized in a similar fashion to hat sizes. Thus, wider open end 104 may have a size that is the same as a hat size of 7.25. Thus, apparel 100 may come in a plurality of sizes capable of snugly fitting on a patient's head, the wider open end varying in the circumference of its aperture based on the size. In other embodiments, illustrated by FIGS. 3a-3b and described in greater detail below, wider open end 104 may be surrounded by a flexible band or drawstring means. In such other embodiments, surgical apparel 100 need only be manufactured in one size, with the flexible band or drawstring means adjusting the circumference of wider open end 104 to accommodate patient heads of varying sizes.

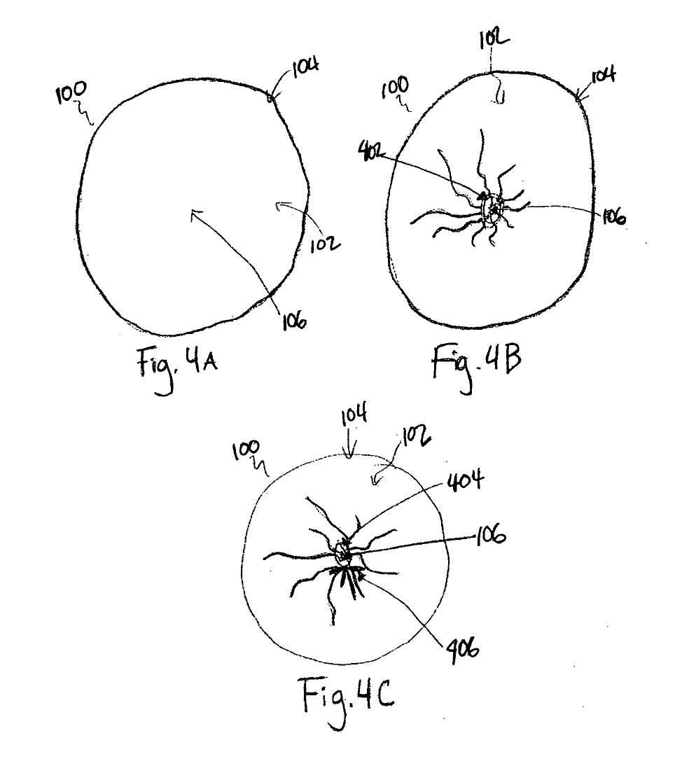

As is shown, narrower closed end 106 of apparel 100 may or may not comprise an aperture. In one embodiment, shell 102 may be contiguous fabric, the contiguous fabric having no apertures except wider open end 104, wider open end 104 formed by the outer boundary of shell 102. Thus, in such an embodiment, narrower closed end 106 may not include an aperture. This embodiment is illustrated by FIG. 4a and is described in greater detail below. In other embodiments, narrower closed end 106 may comprise an aperture. The aperture of the narrower closed end 106 may usually be closed, thus reducing heat loss to the greatest possible extent. To achieve access to a portion of the patient's head, however, the aperture of the narrower closed end 106 may be opened. If the aperture of the narrower closed end is surrounded by a flexible band or drawstring means, the band may be stretched or the means adjusted, allowing access to the portion of the patient's head while remaining as tight as possible, thus reducing heat loss through the patient's head to the greatest possible extent. FIGS. 4b-4c illustrate such narrower closed ends 106 having openable apertures, those figures described in greater detail below.

In various embodiments, at least a part of the surgical apparel 100 may be sterilized to facilitate safer surgical use of the surgical apparel 100. One, some, or all of shell 102's layers of fabric, flexible band(s), and/or drawstring means may be subject to a sterilizing substance or process. In some embodiments, each part of apparel 100 may be subject to a different sterilizing substance/process. Apparel 100 or part(s) thereof may be sterilized by application of any sterilizing substance known in the art, such as an alcohol, a liquid, an aerosol, a cream, or a jelly, or by subjugation to any sterilizing process known in the art, such as exposure to an extreme temperature. In some embodiments surgical apparel 100 may be sterilized by either or both of an apparel 100 manufacturer and/or a medical facility.

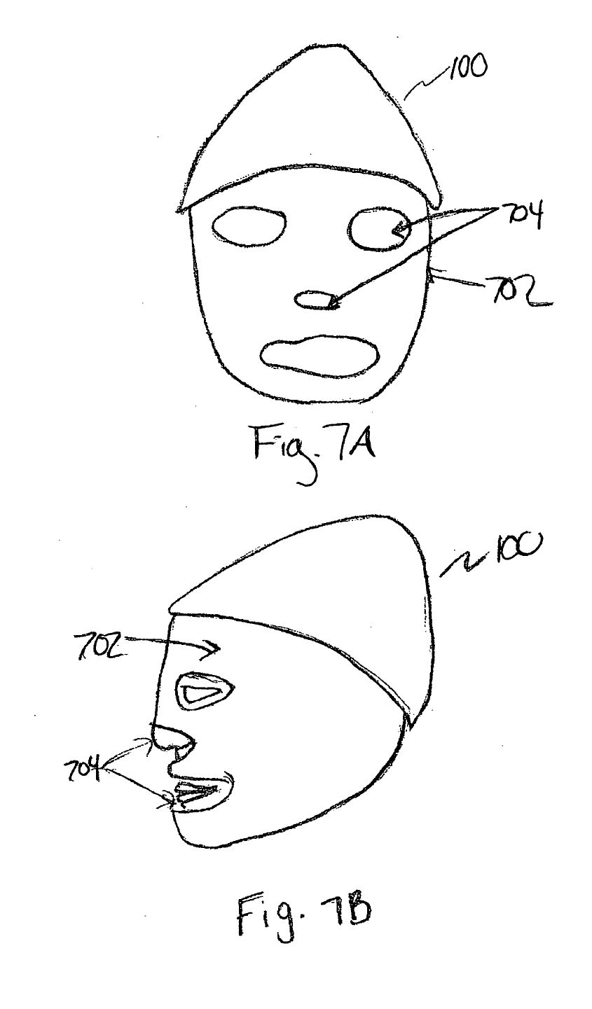

Also, in various embodiments, surgical apparel 100 may be attached to one or both of ear-covering shell attachments and a facial covering, as illustrated in FIGS. 6-8 and described in greater detail below. The shell attachments and/or facial covering may contribute to reducing heat loss through the patient's head during a surgical procedure by covering additional surfaces of the patient's head.

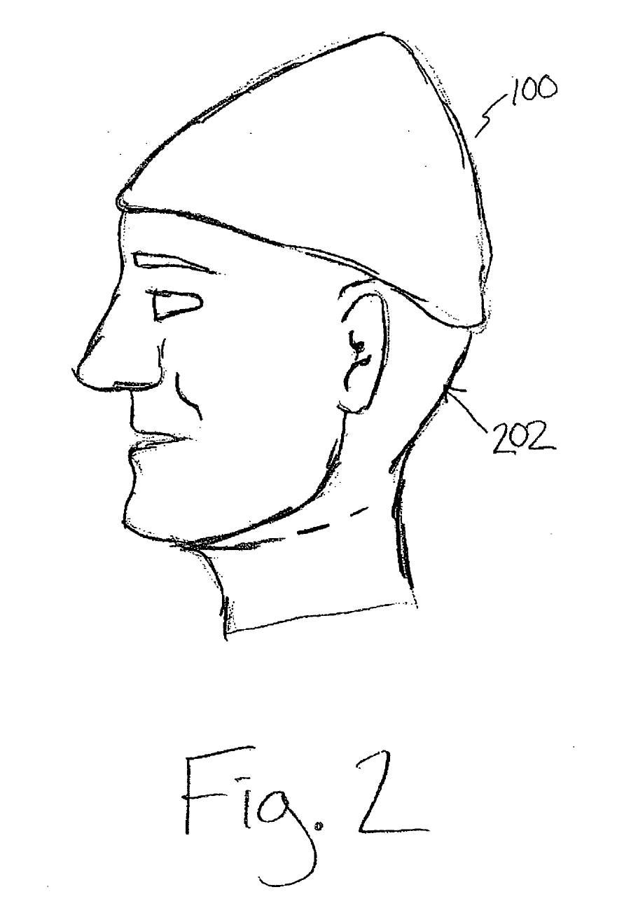

FIG. 2 illustrates the surgical apparel being worn by a surgical patient, in accordance with various embodiments. As is shown, surgical apparel 100 may cover the crown area of a patient's head 202. The crown area may be the same head area covered by, for example, a ski hat or a baseball cap. In other embodiments, apparel 100 may cover more or less of head 202 than is shown. In various embodiments, surgical apparel 100 may be placed on the patient's head 202 either immediately before or some time before a surgical procedure, depending upon a target body heat a physician may want to maintain or achieve. Also, as described above, apparel 100 may be sized to accommodate a wide range of patient head 202 sizes.

FIGS. 3a-3b illustrate surgical apparel having a flexible band or drawstring means surrounding a wider open end of the surgical apparel's shell, in accordance with various embodiments.

As shown in FIG. 3a, the wider open end 104 of surgical apparel 100 may be surrounded by a flexible band 302, allowing apparel 100 to accommodate patient heads of varying sizes while ensuring a snug fit of apparel 100 over the crown area of a patient's head. In various embodiments, flexible band 302 may be made of any material having an elastic property known in the art, such as rubber. In an unstretched position, the flexible band 302 may be much smaller than a typical adult human head, if apparel 100 is intended for an adult human patient, much smaller than a typical child human head, if surgical apparel 100 is intended for a child patient, or much smaller than a typical infant human head, if surgical apparel 100 is intended for an infant patient. In a maximally stretched position, flexible band 302 may be substantially larger than an adult, child, or infant human head, depending on whether apparel 100 is intended for an adult, child, or infant patient. Flexible band 302 may surround the exterior of the fabric comprising the shell 102 of apparel 100 at the wider open end 104, either on the shell 102 surface not in contact with the patient's head, or on the shell 102 surface in contact with the patient's head. In other embodiments, flexible band 302 may be integrated into shell 102, either between layers of fabric or within a layer of fabric.

As illustrated by FIG. 3b, the wider open end 104 of surgical apparel 100 may be surrounded by a drawstring means 304, allowing apparel 100 to accommodate patient heads of varying sizes while ensuring a snug fit of apparel 100 over the crown area of a patient's head. In various embodiments, drawstring means 304 may be made of any material, including the fabric comprising shell 102. Drawstring means 304 need not be an elastic material, but may instead comprise either a tightening means 306 or excess length 306 capable of being tied in a knot to fix a size for apparel 100. If comprising excess length 306, drawstring means 304 may comprise a long, strip whose two ends are not attached in a band. Tightening means 306, on the other hand, may comprise any device capable of substantially maintaining drawstring means 304 in a fixed position. In some embodiments, the drawstring means 304 may have a length such that it may be tightened by tightened by tightening means 306 or tied with excess length 306 to accommodate human heads of a very large size or very small sizes. In one embodiment, where apparel 100 has different shell 102 sizes for adult, child, and infant heads, drawstring means may comprise a different length for each of those head types. Drawstring means 304 may surround the exterior of the fabric comprising the shell 102 of apparel 100 at the wider open end 104, either on the shell 102 surface not in contact with the patient's head, or on the shell 102 surface in contact with the patient's head. In other embodiments, drawstring means 304 may be integrated into shell 102, either between layers of fabric or within a layer of fabric.

FIGS. 4a-4c illustrate top views of various embodiments of the surgical apparel. As illustrated by FIG. 4a, the narrower closed end 106 of surgical apparel 100 may not have an aperture. In such embodiments, narrower closed end 106 may not be openable to allow access to part of a surgical patient's head. Shell 102 may comprise a contiguous sheet of fabric in such embodiment, with the outer bound of shell 102 being wider open end 104. By not including an aperture for narrower closed end 106, apparel 100 may be capable of reducing heat loss through diffusion and air flow to the maximum possible extent.

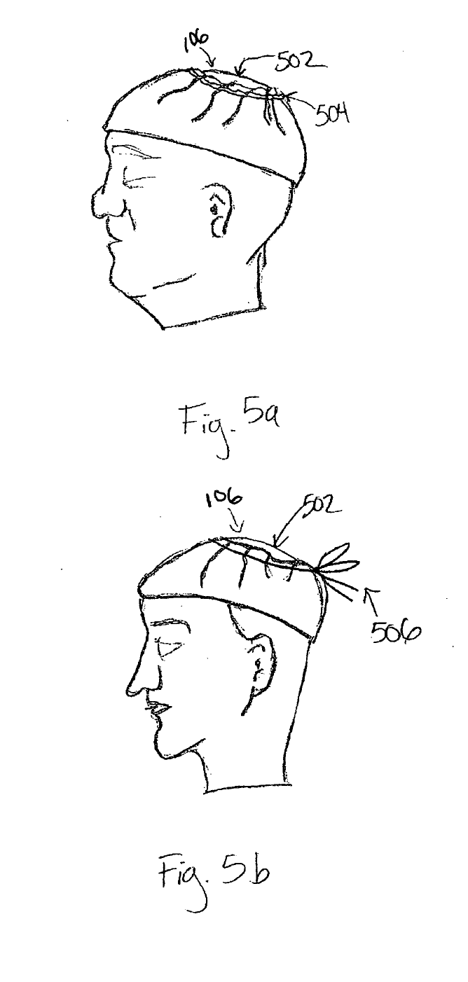

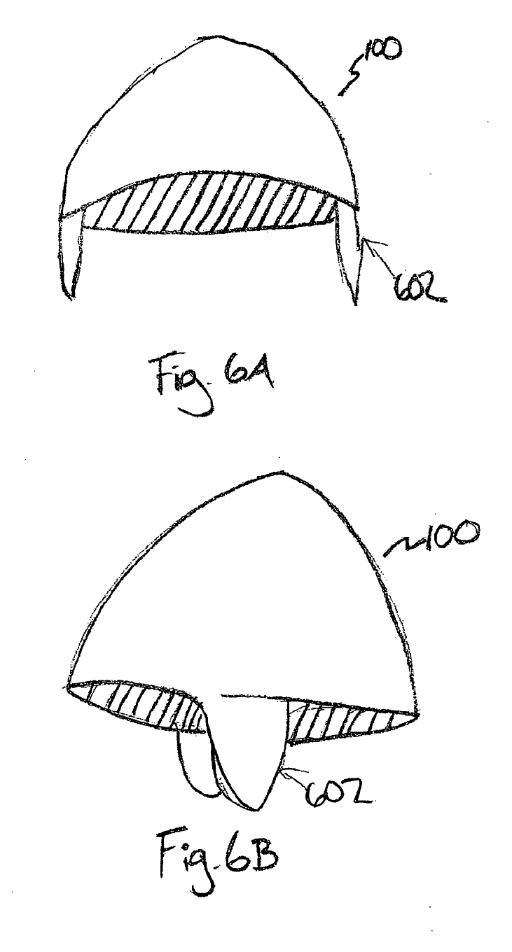

In various embodiments, illustrated by FIG. 4b, narrower closed end 106 may comprised an aperture. The aperture may be completely closed, as shown in FIG. 4b, or may be stretched open, as shown in FIG. 5a. In some embodiments, narrower closed end 106 may be surrounded by a flexible band 402. Flexible band 402 may be made of any material having an elastic property known in the art, such as rubber. In an unstretched position, flexible band 402 may ensure that the aperture of narrower closed end 106 is substantially closed to reduce heat loss. In some embodiments, flexible band 402 may be stretched to a substantially greater circumference. For example, flexible band 402 may be stretched to the same circumference as wider open end 104. In stretching flexible band 402, the aperture of narrower closed end 106 is also opened, as shown in FIG. 5a, allowing access to some of the patient's head. When stretched, flexible band 402 may, however, remain tight, ensuring reduction of heat loss through the aperture of narrower closed end 106 to the greatest extent possible. Also, flexible band 402 may surround the exterior of the fabric comprising the shell 102 of apparel 100 at the narrower closed end 106, either on the shell 102 surface not in contact with the patient's head, or on the shell 102 surface in contact with the patient's head. In other embodiments, flexible band 402 may be integrated into shell 102, either between layers of fabric or within a layer of fabric.

In a number of embodiments, illustrated by FIG. 4c, narrower closed end 106 may comprised an aperture. The aperture may be completely closed, as shown in FIG. 4c, or may be stretched open, as shown in FIG. 5b. In some embodiments, narrower closed end 106 may be surrounded by a drawstring means 404. Drawstring means 404 may be made of any material, including the fabric comprising shell 102. Also, drawstring means 404 need dot be an elastic material, but may instead comprise either a tightening means 406 or excess length 406 capable of being tied in a knot. If comprising excess length 406, drawstring means 404 may comprise a long strip whose two ends are not attached in a band. Tightening means 406, on the other hand, may comprise any device capable of substantially maintaining drawstring means 404 in a fixed position. In some embodiments, the drawstring means 404 may have a length such that it may be tightened by tightened by tightening means 306 or tied with excess length 306 to accommodate human heads of a very large size or very small sizes. In one embodiment, where apparel 100 has different shell 102 sizes for adult, child, and infant heads, drawstring means 404 may comprise a different length for each of those head types. In a maximally drawn/tightened position, drawstring means 404 may ensure that the aperture of narrower closed end 106 is substantially closed to reduce heat loss. In some embodiments, drawstring means 404 and tightening means/excess length 406 may be adjusted to create a substantially greater circumference for the aperture of narrower closed end 106. For example, drawstring means 404 and tightening means/excess length 406 may be adjusted make the aperture of the narrower closed end 106 the same circumference as wider open end 104. Even when adjusted, however, drawstring means 404 and tightening means/excess length 406 may, however, remain tight, ensuring reduction of heat loss through the aperture of narrower closed end 106 to the greatest extent possible. Also, drawstring means 404 may surround the exterior of the fabric comprising the shell 102 of apparel 100 at the narrower closed end 106, either on the shell 102 surface not in contact with the patient's head, or on the shell 102 surface in contact with the patient's head. In other embodiments, drawstring means 404 may be integrated into shell 102, either between layers of fabric or within a layer of fabric.

FIGS. 5a-5b illustrate the narrower closed end of embodiments of the surgical apparel in a more open state to facilitate access to an area of the patient's head. As illustrated by FIGS. 5a-5b, narrower closed end 106 may comprise an opened aperture allowing access to a part of the patient's head 502. Such access may facilitate a surgery requiring access to that area 502 of the head, such as a brain surgery. FIG. 5a shows an embodiment where a flexible band 504 surrounds narrower closed end 106. Flexible band 504 may be the same band as flexible band 402, except in a more stretched position. FIG. 5b shows an embodiment where a drawstring means 506 surrounds narrower closed end 106. Drawstring means 506 may be the same means as drawstring means 404, except in an adjusted position.

FIGS. 6a-6b illustrate surgical apparel having ear-covering shell attachments, in accordance with various embodiments. FIG. 6a illustrates apparel 100 with ear-covering shell attachments 602 from a front/back position, and FIG. 6b illustrates the same apparel 100 and attachments 602 from a side position. In various embodiments, ear-covering shell attachments 602 may be made of the same or a different fabric then apparel 100, and may have the same or a different number of layers of fabric as apparel 100. In one embodiment, attachments 602 are part of apparel 100 and shell 102, contiguous with shell 102. In other embodiments, attachments 602 are not part of apparel 100 but are attached to apparel 100 through any means known in the art, such as Velcro, snaps, buttons, zippers, strings/laces, and glue. Ear-covering shell attachments 602 may be capable of reducing heat loss from the patient's ears to the ambient through air flow or diffusion.

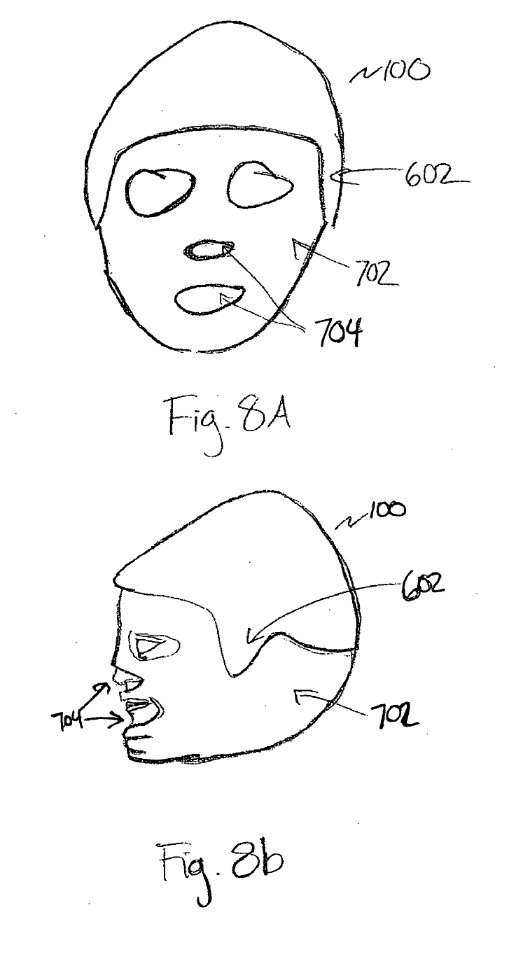

FIGS. 7a-7b illustrate surgical apparel having an attached facial covering, in accordance with various embodiments. FIG. 7a illustrates apparel 100 with a facial covering 702 from a front position, and FIG. 7b illustrates the same apparel 100 and covering 702 from a side position. In various embodiments, facial covering 702 may be made of the same or a different fabric then apparel 100, and may have the same or a different number of layers of fabric as apparel 100. In one embodiment, covering 702 is part of apparel 100 and shell 102, contiguous with shell 102. In other embodiments, covering 702 is not part of apparel 100 but is attached to apparel 100 through any means known in the art, such as Velcro, snaps, buttons, zippers, strings/laces, and glue. Facial covering 702 may be capable of reducing heat loss from the patient's face to the ambient through air flow or diffusion. In one embodiment, facial covering may have apertures 704 for patient eyes, nose, and mouth cut out from covering 702, allowing the patient to breath, speak, and interact with the environment. Facial covering 702 may cover only the patient's face, or may cover the entire part of the patient's head not covered by apparel 100.

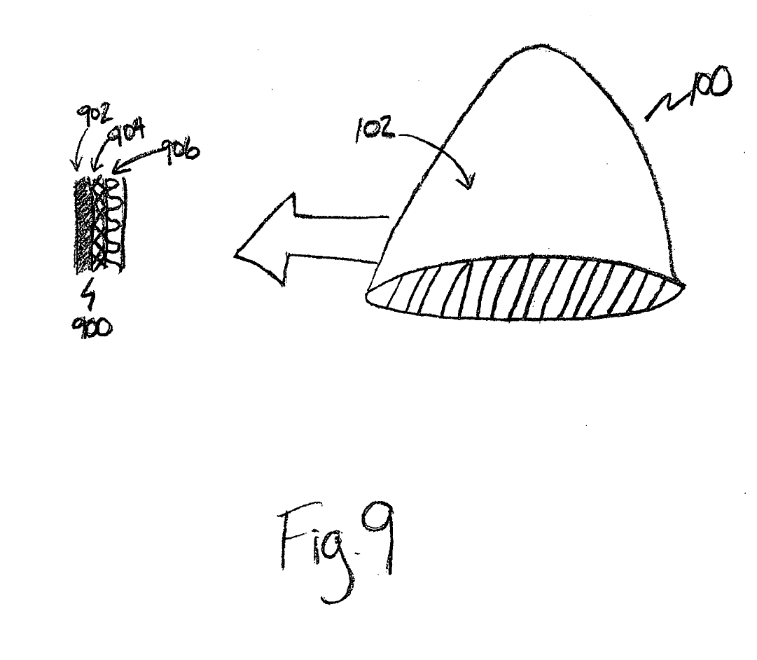

FIGS. 8a-8b illustrate surgical apparel having both ear-covering shell attachments and an attached facial covering, in accordance with various embodiments. FIG. 8a illustrates apparel 100 with ear-covering shell attachments 602 and a facial covering 702 from a front position, and FIG. 8b illustrates the same apparel 100, shell attachments 602, and covering 702 from a side position.

FIG. 9 illustrates a cross section view of a shell of the surgical apparel, in accordance with various embodiments. As is shown, apparel 100 may be comprised of a shell 102. A cross section 900 of shell 102 may show a plurality of layers of fabric, such as layers 902, 904, and 906. Layers of fabric 902-906, may be of any of the different fabric types discussed above, and may have any sort of thickness. Each layer 902-906 may have a fabric type different from one or all of the other layers 902-906, and/or a thickness different from one or all of the other layers 902-906. In other embodiments all layers 902-906 may have the same fabric type and/or the same thickness. In yet other embodiments, shell 102 may be comprised of more or fewer layers than layers 902-906.

FIG. 10 illustrates surgical apparel dimensioned to cover all of a surgical patient's hair, in accordance with various embodiments. As illustrated, all of the surgical patient's hair is covered by apparel 100. Covering all of the patient's hair may be a requirement in many hospitals and operating rooms, and may further enhance the ability of apparel 100 to reduce heat loss to the ambient during a surgical procedure.

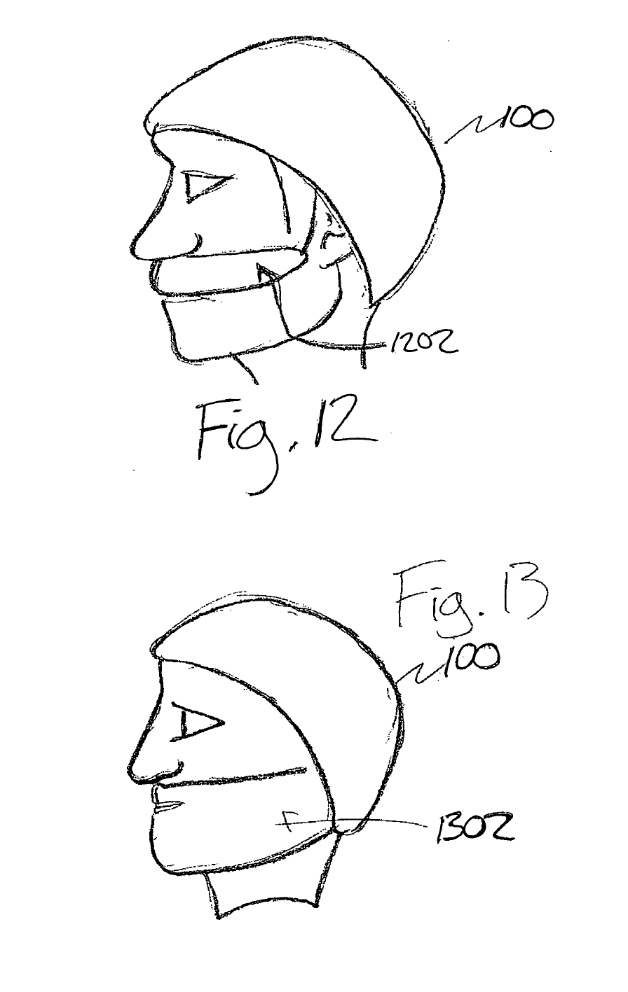

FIG. 11 illustrates surgical apparel having an attached beard covering, in accordance with various embodiments. As illustrated, beard covering 1102 may be attached to apparel 100 in such a way as to cover a patient's beard. It may be desirable to cover the patient's beard, as it may be a requirement in many hospitals and operating rooms to cover patient hair, including a beard, during a surgical procedure. In various embodiments, beard covering 1102 may be made of the same or a different fabric then apparel 100, and may have the same or a different number of layers of fabric as apparel 100. In one embodiment, covering 1102 is part of apparel 100 and shell 102, contiguous with shell 102. In other embodiments, covering 1102 is not part of apparel 100 but is attached to apparel 100 through any means known in the art, such as Velcro, snaps, buttons, zippers, strings/laces, and glue. Beard covering 1102 may be capable of reducing heat loss from the patient's face to the ambient through air flow or diffusion.

FIG. 12 illustrates surgical apparel having an attached mustache covering, in accordance with various embodiments. As illustrated, mustache covering 1202 may be attached to apparel 100 in such a way as to cover a patient's mustache. It may be desirable to cover the patient's mustache, as it may be a requirement in many hospitals and operating rooms to cover patient hair, including a mustache, during a surgical procedure. In various embodiments, mustache covering 1202 may be made of the same or a different fabric then apparel 100, and may have the same or a different number of layers of fabric as apparel 100. In one embodiment, covering 1202 is part of apparel 100 and shell 102, contiguous with shell 102. In other embodiments, covering 1202 is not part of apparel 100 but is attached to apparel 100 through any means known in the art, such as Velcro, snaps, buttons, zippers, strings/laces, and glue. Mustache covering 1202 may be capable of reducing heat loss from the patient's face to the ambient through air flow or diffusion.

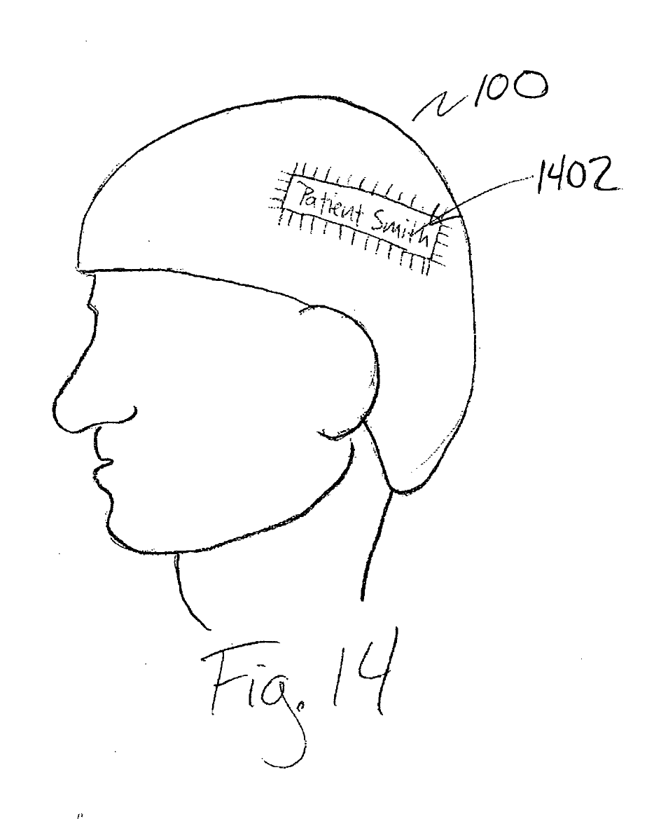

FIG. 13 illustrates surgical apparel having attached beard and mustache coverings, in accordance with various embodiments. As illustrated, beard and mustache coverings, such as those described above in reference to FIGS. 11 and 12, may be combined into one covering 1302, which may be attached to apparel 100 in the same manner. In other embodiments, both coverings may be simultaneously attached to apparel 100, but may not be combined into one covering. In one embodiment, beard and facial covering 1302 may have an aperture for the patient's mouth cut out from covering 1302, allowing the patient to breath, speak, and interact with the environment.

FIG. 14 illustrates surgical apparel having an attached patient identifier, in accordance with various embodiments. As illustrated, a patient identifier 1402 may be attached to the shell 102 of apparel 100. The identifier 1402 may be made of any material known in the art. Identifier 1402 may be made of a fabric, which may be the same or a different fabric from shell 102, and may be attached through an adhesive, may be stitched on, or may be attached in any manner known in the art. In other embodiments, identifier 1402 may be an adhesive strip, such as tape, and may be adhered to apparel 100. Identifier 1402 may also have identifying information regarding the surgical patient, such as a name, patient identification number, or some other type of identifier. The identifying information may be added to identifier 1402 in any manner known in the art. The information may be written on with a marker, pen or pencil, may be typed on, or may be stitched on with thread. Patient identification may be greatly aid in ensuring patient safety, especially in an operating room, to provide an additional check for determining whether the patient is the correct patient, is having the correct procedure, etc.

Although specific embodiments have been illustrated and described herein, it will be appreciated by those of ordinary skill in the art that a wide variety of alternate and/or equivalent implementations may be substituted for the specific embodiments shown and described, without departing from the scope of the embodiments of the present invention. This application is intended to cover any adaptations or variations of the embodiments discussed herein. Therefore, it is manifestly intended that the embodiments of the present invention be limited only by the claims and the equivalents thereof.

1-23. (canceled)

24. A method of retaining body heat in a surgical patient during a surgical procedure, the method comprising:
providing a surgical apparel comprising a fabric shell having a closed end and an open end dimensioned to fit over a crown area of a head of a surgical patient, and one or more layers of fabric having a low thermal transfer coefficient adapted to complement the closed end to reduce an amount of heat transfer from a head of a surgical patient to ambient during a surgical procedure on a surgical patient wearing the surgical apparel; and
during a surgical procedure, covering the crown area of the head of the surgical patient with the surgical apparel.

25. The method of claim 24, further comprising applying the surgical apparel to the crown area of the head of the surgical patient prior to initialization of the surgical procedure.

26. The method of claim 24, further comprising disposing of the surgical apparel subsequent to the surgical procedure on the surgical patient.

27. The method of claim 24, wherein the providing a surgical apparel further comprises providing a substantially conically shaped fabric shell.

28. The method of claim 27, wherein the providing a substantially conically shaped fabric shell further comprises providing the open end wider than the closed end.

29. The method of claim 24, further comprising at least partially opening the closed end of the fabric shell.

30. The method of claim 24, wherein the providing a fabric shell further comprises providing at least two layers of fabric, the two layers comprising different fabric types, and one comprising the low thermal transfer coefficient.

31. The method of claim 24, further comprising sterilizing at least one of the one or more layers of fabric of the shell prior to the surgical procedure on the surgical patient.

32. The method of claim 24, wherein the providing a surgical apparel further comprises providing a shell that is dimensioned to cover the surgical patient's hair.

33. The method of claim 24, wherein the providing a surgical apparel further comprises providing a patient identifier tag attached to the shell and identifying the surgical patient.

34. A method of retaining body heat in a surgical patient during a surgical procedure, the method comprising:
with a surgical apparel comprising a substantially conically shaped shell having a narrower closed end and a wider open end dimensioned to fit over a crown area of a head of a surgical patient, and one or more layers of fabric having a low thermal transfer coefficient adapted to reduce an amount of heat transfer from the head of the surgical patient to the ambient during a surgical procedure on a patient wearing the surgical apparel, fitting the surgical apparel over the crown area of the head of a surgical patient during a surgical procedure on the surgical patient.

35. The method of claim 34, wherein the wider open end of the shell further comprises a means for securing the shell about the head of a surgical patient.

36. The method of claim 34, wherein the narrower closed end of the shell further comprises a closable aperture, and further comprising a means for at least partially closing the aperture during a surgical procedure.

37. The method of claim 34, further comprising disposing of the surgical apparel subsequent to the surgical procedure on the surgical patient.

38. The method of claim 34, wherein the shell is comprised of at least two layers of fabric, the two layers having different fabric types, and at least one of the different fabric types further comprising the low thermal transfer coefficient.

39. The method of claim 37, wherein the surgical apparel further comprises ear-covering shell attachments comprised of one or more layers of fabric, the shell attachments attached to the shell at positions allowing the shell attachments to cover ears of the surgical patient and contributing to reducing the amount of heat transfer from the head of the surgical patient to the ambient during a surgical procedure.

40. The method of claim 37, wherein the surgical apparel further comprises a facial covering comprised of one or more layers of fabric, the facial covering attached to the shell at positions allowing the facial covering to cover the surgical patient's face and contributing to reducing the amount of heat transfer from the head of the surgical patient to the ambient during a surgical procedure.

41. A method of retaining body heat in a surgical patient during a surgical procedure, the method comprising:
providing a surgical apparel comprising a substantially conically shaped shell having a narrower closed end and a wider open end dimensioned to fit over a crown area of a head of a surgical patient, and one or more layers of fabric having a low thermal transfer coefficient adapted to complement the closed end to reduce an amount of heat transfer from the head of the surgical patient to the ambient during a surgical procedure;
one of a flexible band and a drawstring means surrounding the wider open end to facilitate snug fits for surgical patient heads of varying sizes;
one of:
a flexible band surrounding a closed aperture of the narrower closed end, the flexible band to facilitate opening of the end, and the flexible band becomes or remains tight when expanded, reducing the amount of heat transfer from the head of the surgical patient to the ambient during a surgical procedure, or
a drawstring means surrounding the closed aperture of the narrower closed end, the drawstring means to facilitate opening of the end, and the drawstring means remains partially drawn even when loosened, reducing the amount of heat transfer from the head of the surgical patient to the ambient during a surgical procedure;
initially at least partially covering the crown area of the head of the surgical patient with the surgical apparel and snugly fitting the wider open end to the head of the surgical patient prior to a surgical procedure; and
disposing of the surgical apparel subsequent to the surgical procedure on the surgical patient.

42. The method of claim 41, wherein providing a surgical apparel further comprises providing a plurality of layers of fabric wherein at least one layer further comprises the low thermal transfer coefficient.

43. The method of claim 41, wherein providing a surgical apparel further comprises providing one of: an ear-covering shell attachments attached to the shell at positions allowing the shell attachments to cover ears of the surgical patient and contributing to reducing the amount of heat transfer from the head of the surgical patient to the ambient during a surgical procedure, and a facial covering attached to the shell at a position allowing the facial covering to cover the surgical patient's face and contributing to reducing the amount of heat transfer from the head of the surgical patient to the ambient during a surgical procedure.
